(19) United States
(12) Patent Application Publication
(10) Pub. No.: US 2013/0303930 A1
(43) Pub. Date: Nov. 14, 2013

(54) Game-based incentive spirometer and a method of quantifying and recording performance

(71) Applicants: John Elefteriades, Guilford, CT (US); Joseph Bango, New Haven, CT (US); Michael Dziekan, Bethany, CT (US)

(72) Inventors: John Elefteriades, Guilford, CT (US); Joseph Bango, New Haven, CT (US); Michael Dziekan, Bethany, CT (US)

(21) Appl. No.: 12/804,432

(22) Filed: Jul. 21, 2010

Related U.S. Application Data
(60) Provisional application No. 61/271,625, filed on Jul. 22, 2009.

(51) Int. Cl. A61B 5/087; A61B 5/00; A61B 5/08

(52) U.S. Cl. CPC A61B 5/087; A61B 5/0803; A61B 5/744
USPC 600/538

(57) ABSTRACT

An incentive spirometry device designed to assist patients with respiratory therapy by means of an electronic air flow sensor that provides patients with visual or other positive feedback when they inhale or exhale at a predetermined flow rate or volume and sustain the act for a predetermined minimum time period. The objectives are to increase transpulmonary pressure and inspiratory volumes, improve inspiratory muscle performance, and re-establish or simulate the normal pattern of pulmonary hyperinflation. When the procedure is repeated on a regular basis, airway patency may be maintained and lung atelectasis prevented and reversed. By means of a connection to a personal computer, a video game like therapy session could enable patients to have a more effective and enjoyable session.

### CROSS REFERENCE TO RELATED APPLICATION:

Provisional Application No. 61/271625 was filed on 22 Jul. 2009

### BACKGROUND

1. Field of Invention

Incentive spirometry is designed to mimic natural sighing or yawning by encouraging the patient to take long, slow, deep breaths. This is accomplished by using a device that provides patients with visual or other positive feedback when they inhale at a predetermined flowrate or volume and sustain the inflation for a minimum of 3 seconds. The objectives are to increase transpulmonary pressure and inspiratory volumes, improve inspiratory muscle performance, and re-establish or simulate the normal pattern of pulmonary hyperinflation. When the procedure is repeated on a regular basis, airway patency may be maintained and lung atelectasis prevented and reversed. Incentive spirometry should be contrasted with expiratory maneuvers (such as the use of blow bottles) that do not mimic the sigh and have been associated with the production of reduced lung volumes. The described method of Incentive Spirometry details how to utilize a personal computer to provide accurate visual feedback as well as a means of tailoring a patient-specific Incentive Spirometry routine, and providing a quantitative method of recording data.

2. Background Description of Prior Art

Traditional Incentive Spirometry devices are small plastic devices with either single or multiple plastic balls contained within partially sealed tubes. When using an incentive spirometer, the purpose is to help the patient to recover more quickly from invasive surgery, and prevent pulmonary problems. Although the traditional Incentive Spirometry devices are simple and fairly easy to use, the results are dependant upon the patient's proper use of the device. If a patient is shown how to properly utilize an Incentive Spirometer, then good results should be expected; however, if a patients does not utilize the Incentive Spirometer in the proper manner, then its effectiveness will be compromised. Several factors can cause the effectiveness of an Incentive Spirometer to be compromised. Some of the factors that could contribute to decreased effectiveness are:
Holding the Incentive Spirometer at an angle
Having the patient performing at a nearly prone position
Having the patient hold their breath for too short an amount of time
Having the patient hold their breath for too long an amount of time
Having the patient fail to exhale completely
Having the patient inhale or exhale too slowly
Having the patient inhale or exhale too quickly
Delaying the start of Incentive Spirometry therapy

Any of these factors could result in decreased effectiveness of an Incentive Spirometer. A patient should not use incentive spirometry if they can't understand or demonstrate proper use of the device. If the Incentive Spirometer is not held in an upright position, it will give inaccurate results. A tilted flow-oriented device requires less effort to raise the balls or discs; a volume-oriented device will not function correctly unless upright. With a traditional Incentive Spirometer, there is no accurate means of determining the rate of inhalation or exhalation, as well as no accurate means of determining the time a patient's breath is held. The described invention outlines how to construct an Incentive Spirometer and utilize a personal computer to quantify and record data. A custom, patient-specific therapy can be implemented easily to help a patient avoid pulmonary complications.

Previous iterations of Incentive Spirometers have no means of accurately quantifying results, and the only means is usually the patient describing to the doctor or care-giver what they “feel” or “want” the results to be. As we have all learned throughout life, people lie—especially if those people happen to be patients. Whether those lies are intentional or blatant, the information that a doctor or care-giver has at their disposal to evaluate a patients progress is limited, or can be erroneous is the patient lies or exaggerates. In addition, older patients may not be fully capable of quantifying an accurate amount of time for holding ones breath. What one person may think is three to five seconds may in reality be only one or two seconds. The described invention will give accurate results since it is not a person's estimation of elapsed time, but an accurate computer measurement of elapsed time. As Albert Einstein once said when describing the relative nature of time—“Put your hand on a hot stove for a minute, and it seems like an hour. Sit with a pretty girl for an hour, and it seems like a minute. That's relativity.” When it comes to doing difficult tasks, time is truly relative. If a person is doing push-ups or sit-ups, the amount of time passing will seem much slower when the same person is watching their favorite television show for the equivalent amount of time. Human beings are too subjective when it comes to measuring time or intervals of time. A computer has no subjectivity, and therefore is an accurate indicator of time and elapsed time. Along with the subjective comprehension of time, humans are also fairly subjective when they are quantifying an amount of effort or physical work. If one person asks another how much work they did during a typical day at their job, a reply will be returned indicating a usually over-exaggerated degree of effort. On rare occasions, a reply will be returned to the same question indicating an under-exaggerated degree of effort. What is needed is an impartial method of determining effort. Computers can accurately quantify both time and effort without any subjectivity. The described invention utilizes the combination of a personal computer and an appropriate airflow sensor to transform the patient's spirometric data into a visual representation on a personal computers screen. It should be obvious to those skilled in the art that a integrated combination of a personal computer-like device can accomplish the same task; however, the preferred embodiment of the described invention will utilize an airflow measuring transducer that is connects the patients airflow to an electrical signal suitable for input to a personal computer. The benefit of the described invention is that it is easy to use, and combines a video game feel to an otherwise boring task. The combination of a video game response and recordable progress will allow patients to alleviate any pulmonary conditions quickly and effectively.

### BRIEF DESCRIPTION OF THE DRAWINGS

FIG. 1 shows a photograph of a commercially available “Tri-ball” Incentive Spirometer that is typically found in hospitals throughout the world. The device consists of three chambers containing a single plastic ball that respond differently to varying amount of airflow. A short section of flexible tube connects a mouthpiece to the device.

FIG. 2 shows a schematic representation of the described invention consisting of a flexible hose connected to an airflow transducer, which converts airflow information to a corresponding electrical signal, suitably conditioned to transform the data to a personal computer.

FIG. 3 shows a schematic representation of a person will utilize the described Incentive Spirometer. A person blows into the flexible tube which is connected to an integrated airflow transducer and electrical conversion circuit, which allows for communication to a personal computer (not shown).

FIG. 4 shows a schematic representation of the integrated Incentive Spirometry device which is connected to an attached personal computer.

FIG. 5 shows a screenshot of the working prototype developed by Connecticut Analytical Corporation showing the computer animated background. The screenshot also shows a small space ship that responds to the amount of airflow that is converted by the airflow transducer. The screenshot also details patient-specific information. In this image, the space ship is shown “flying” at the bottom of the screen since no airflow is being measured by the airflow transducer. As more breath is exhaled or inhaled, the space ship will rise to the top of the screen. As less breath is exhaled or inhaled, the space ship sinks to a lower position on the screen.

FIG. 6 shows a screenshot of the working prototype developed by Connecticut Analytical Corporation showing the computer animated background. The screenshot also shows a small space ship that responds to the amount of airflow that is converted by the airflow transducer. The screenshot also details patient-specific information. In this image, the space ship is shown “flying” between two lines that are “targets” for helping the patient to exert a specific amount of effort to cause the space ship to remain stationary within. As more breath is exhaled or inhaled, the space ship will rise to the top of the screen. As less breath is exhaled or inhaled, the space ship sinks to a lower position on the screen.

### DETAILED DESCRIPTION OF THE INVENTION

The importance of Incentive Spirometry has been debated, and according to the American College of Chest Physicians, an article was written entitled “The Effect of Incentive Spirometry on Postoperative Pulmonary Complications”. The relevant text cited is as follows: “Cardiac and upper abdominal surgical procedures are associated with a high incidence of postoperative pulmonary complications (PPCs), which are defined as pulmonary abnormalities occurring in the postoperative period producing clinically significant, identifiable disease or dysfunction that adversely affects the clinical course. The incidence rate depends on the surgical site, the presence of risk factors, and the criteria used to define a PPC. Reported incidence rates for upper abdominal surgery range from 17 to 88%. The basic mechanism of PPCs is a lack of lung inflation that occurs because of a change in breathing to a shallow, monotonous breathing pattern without periodic sighs, prolonged recumbent positioning, and temporary diaphragmatic dysfunction. Mucociliary clearance also is impaired postoperatively, which, along with the decreased cough effectiveness, increases risks associated with retained pulmonary secretions. Ward et al showed that postoperative atelectasis is better reduced by taking a deep breath and holding it for 3 s than by taking multiple deep breaths or not holding a deep breath. The first reports on the use of such sustained maximal inspirations for the treatment of postsurgical patients originated in Great Britain. The first major study showing the benefits of postoperative maximal inspiration was carried out by Thoren in 343 patients who were undergoing cholecystectomy. Thoren documented an incidence of atelectasis (detected via radiograph) of 42% in control subjects vs 27% in patients treated postoperatively with physical therapy including deep-breathing (DB) exercises. The incidence rate declined further, to 12%, in patients who received additional preoperative instruction in the breathing exercises. An Incentive Spirometer is a device that encourages, through visual and/or audio feedback, the performance of reproducible, sustained maximal inspiration. Incentive Spirometry (IS) is the treatment technique utilizing incentive spirometers. Bartlett et al developed an incentive spirometer that both provided visual feedback to the patient and recorded the number of successful breathing maneuvers. This unit, the Bartlett-Edwards incentive spirometer, remained the standard for many years, although it has since been replaced by less expensive, single-use units. The first specific report of Incentive Spirometry as a treatment technique appears to be that of Van de Water et al, who compared Incentive Spirometry to intermittent positive-pressure breathing (IPPB) in 30 patients after they had undergone abdominal bilateral adrenalectomy. No statistical difference was reported in the incidence of pulmonary complications between treatment groups. Incentive Spirometry remains a widely used technique for the prophylaxis and treatment of respiratory complications in postsurgical patients. O'Donohue surveyed its use in the United States and reported that 95% of hospitals in which cardiothoracic and abdominal surgery was performed used Incentive Spirometry in postoperative care. Jenkins and Soutar reported a usage rate of 44% in hospitals in which coronary artery bypass graft (CABG) surgery was carried out in the United Kingdom. More recently, Wattie repeated this survey and found that the usage rate had increased to 71%, despite recent publications that have cast doubt on both the need for Incentive Spirometry in patients undergoing CABG surgery and the effectiveness of Incentive Spirometry in this population.”

The described invention utilizes visual information to quantify airflow measurements, but it is obvious to those skilled in the art that both visual and audible information can also be utilized. The Incentive Spirometer is designed to be utilized in the following settings:
Critical care
Acute care inpatient
Extended care and skilled nursing facility
Home care

The small size makes the described invention easy to utilize in any of these settings. A person could easily implement an effective Incentive Spirometry therapy in the comfort of their own home just as easily as if they were in a hospital or extended care facility. The main feature of Connecticut Analytical's Incentive Spirometer is that it turns an otherwise boring task into a fun game.

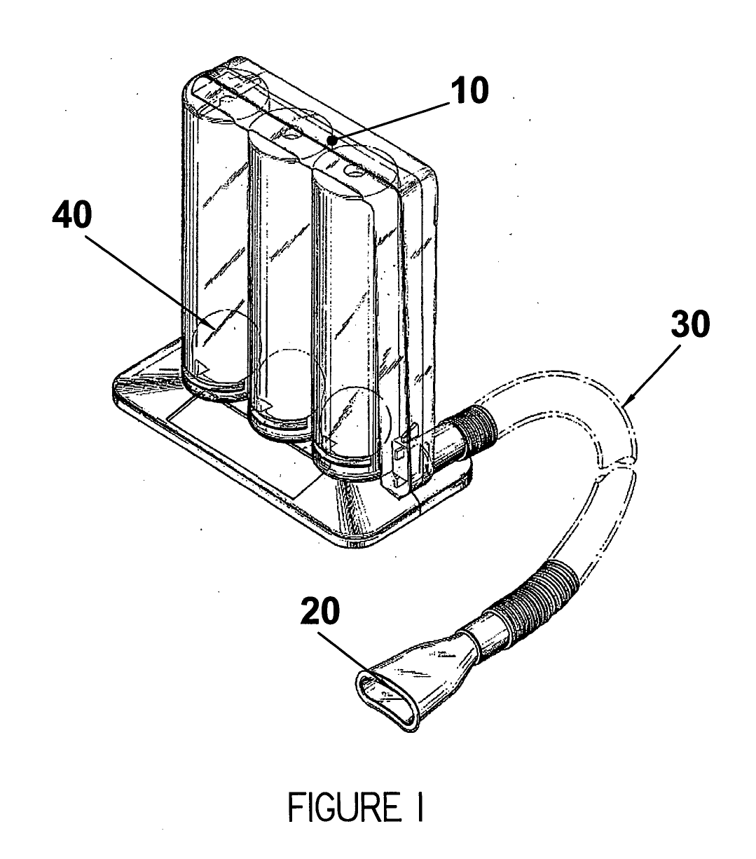

FIG. 1 shows a typical commercially available Incentive Spirometer. A flexible hose 30 connects the mouthpiece 20 to the main section 10. As a person inhales while they have the mouthpiece 20 in their mouth, the rate of airflow will raise the individual plastic balls 40 to the top of their enclosed cylinder. This rate of airflow is pre-calibrated in such a manner as to have all three balls 40 raised to the tops of their enclosed cylinders when the equivalent volume of air inspired is 3000 cubic centimeters in a time of three seconds. The point here is to ensure a full, deep breath from the patient. Although simple and easy to use, the Incentive Spirometer makes an assumption that everyone's lung volume will be equivalent to this. The described invention has the ability to record a baseline lung volume before surgery, and use this stored patient-specific baseline during the post-operative period to give a more accurate assessment of complete inhalation. An adult person, who is four foot tall, will not have the same lung volume as one who is six foot tall. The same applies to two individuals who are the same physical stature, but have different internal physical differences; such as, one is a smoker, while the other is a long distance runner. Differences like these can be incorporated into the therapy regime to give the patient a more effective and customized treatment. This type of Incentive Spirometer has no way of determining of a person has completely evacuated their lungs before they begin to inspire. If the person using this device does not completely exhale, then the amount of air inhaled will not correspond to a full volume of air in the lungs. The described invention has a means of running a baseline to help the patient accurately determine the correct amount of lung volume.

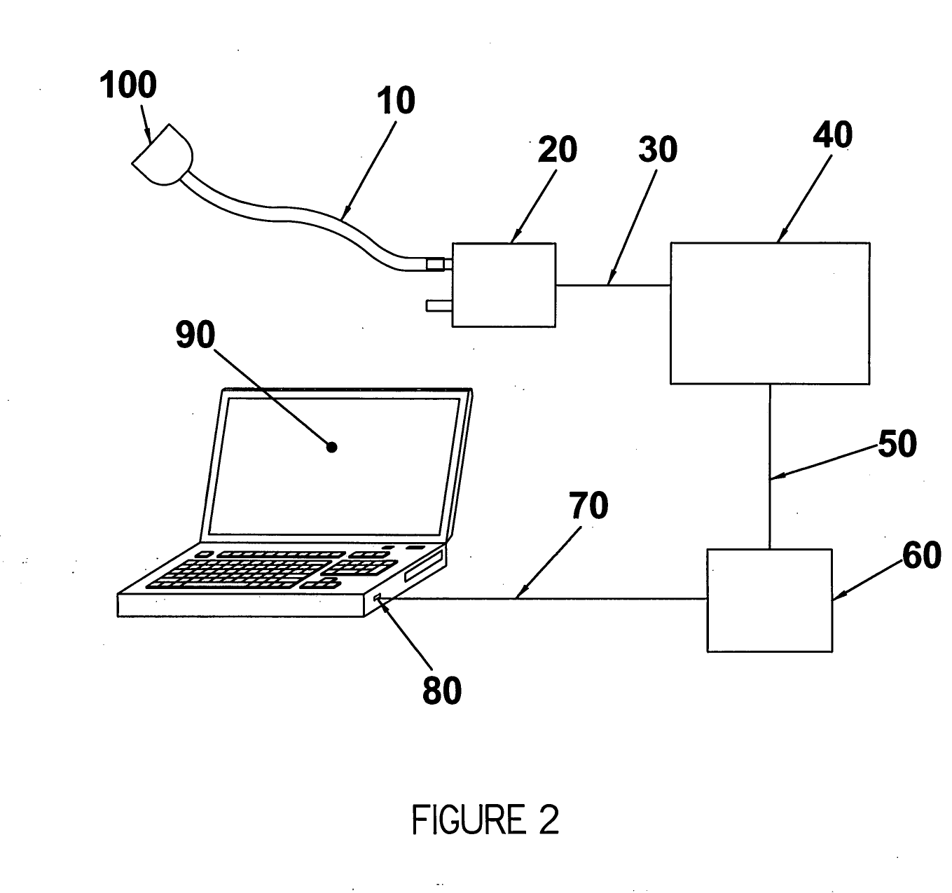

FIG. 2 details a block diagram of the described invention. The Incentive Spirometer consists of a mouthpiece 100 attached to a section of flexible tubing 10 that connects to an airflow transducer 20. The airflow transducer 20 has the capability to convert airflow into an analog voltage output. As the person inhales at a greater rate, the airflow transducer 20 will respond with a corresponding increasing amount of voltage. It is obvious to those skilled in the art that an airflow transducer 20 could be utilized having the opposite property where the increased airflow causes a corresponding decreasing amount of voltage—as long as one knows which transducer is used, the output electronics can compensate accordingly. In the described invention, the airflow transducer 20 will give an increased output with a greater flow rate. The airflow transducer 20 is connected by appropriate wires 30 to an analog to digital converter 40 that also contains a microprocessor. The analog to digital converter 40 functions to convert the analog input voltage to a corresponding digital representation. The converted digital values are sent to an interface circuit 60 by means of appropriate wires 50. The interface circuit will convert the digital representation of the analog voltage sent from the airflow transducer 20 to an appropriate computer interface, such as USB (Universal Serial Bus). The interface circuit 60 connects to the personal computer 90 by means of a typical USB cable 70. The USB cable 70 connects to the personal computer's USB port 80. Although the described invention utilizes a USB interface to communicate with the personal computer 90, it is obvious to those skilled in the art that one of several computer interfaces will work equally well, such as firewire, a serial COMM port utilizing EIA-232 protocol, a serial COMM port utilizing EIA-485 protocol, a parallel printer port, or IrDA (Infrared Data Acquisition). It is also equally possible to utilize a wireless interface between the personal computer and the interface circuit 60. The wireless connection would be comprised of either an optical interface, such as infrared, or RF (Radio Frequency). The described invention will utilize the more common USB port 80 found on virtually every computer. The USB protocol will function with USB 1.1 or USB 2.0 protocol. The airflow transducer 20 has the ability to provide an analog output signal if the patient is performing an inhalation of exhaling, although the signals will be correspondingly opposite in polarity. If the patient is inhaling, the analog signal will be positive, and negative if the patient is exhaling.

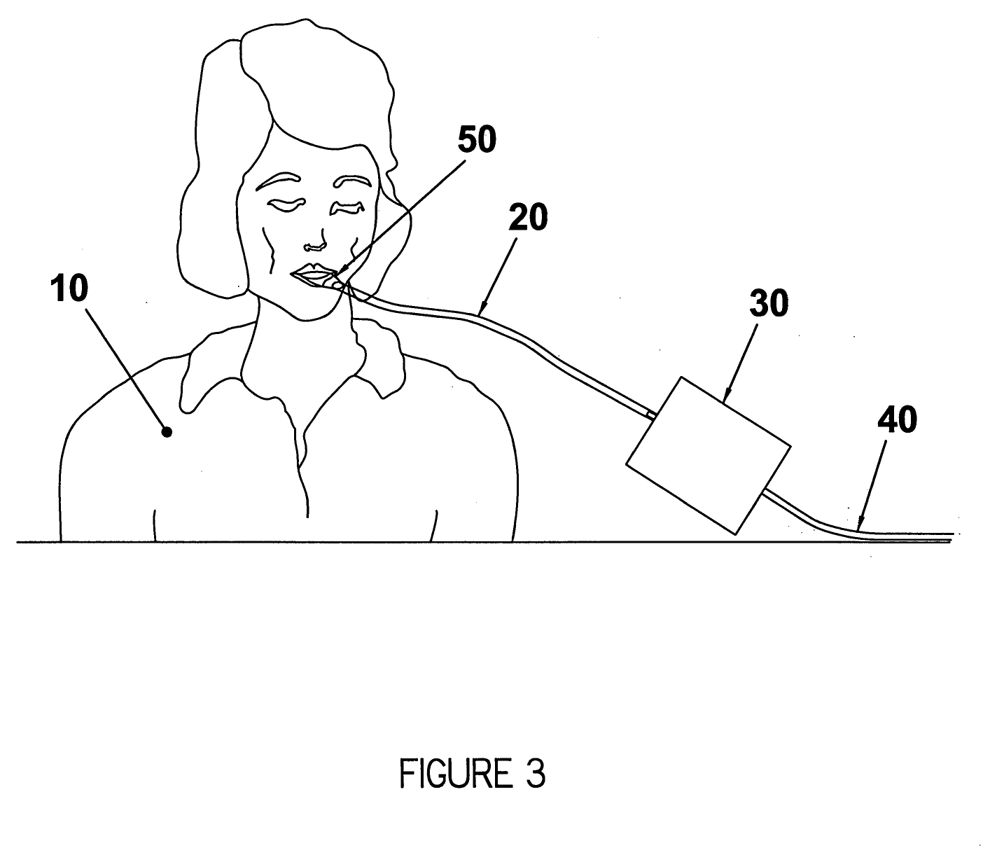

FIG. 3 shows a drawing of a person 10 utilizing the Incentive Spirometer system comprising all the interface electronics and airflow transducer described in FIG. 2 in a small enclosure 30. The person 10 places the mouthpiece 50 in their mouth. The mouthpiece 50 is connected to the integrated Incentive Spirometer 30 by means of a flexible tube 20. The integrated interface circuit of the Incentive Spirometer 30 converts the inhaled airflow to a corresponding digital representation that has been formatted for USB communication and is connected to the personal computers (not shown) USB port by means of a USB cable 40.

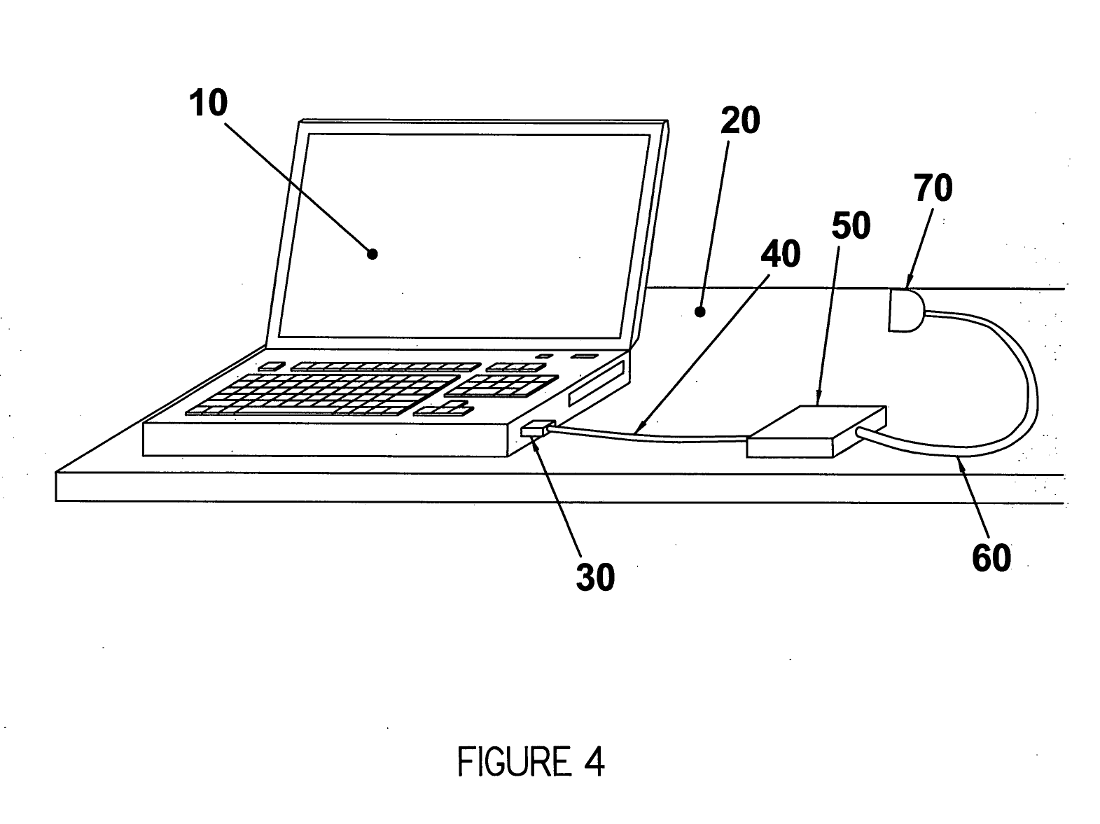

FIG. 4 details the complete Incentive Spirometer 50 shown connected to a personal computer 10 USB port 30 by means of a USB cable 40. The flexible tube 60 connects the mouthpiece 70 to the Incentive Spirometer 50. The whole system is shown on the top of a table 20. Although a contemporary laptop is shown for the personal computer 10, any contemporary desktop or tower computer can be utilized, provided it has at least one USB port and the proprietary software required for use with the Incentive Spirometer. The proprietary software can be preloaded onto the personal computer or it can be installed by the user.

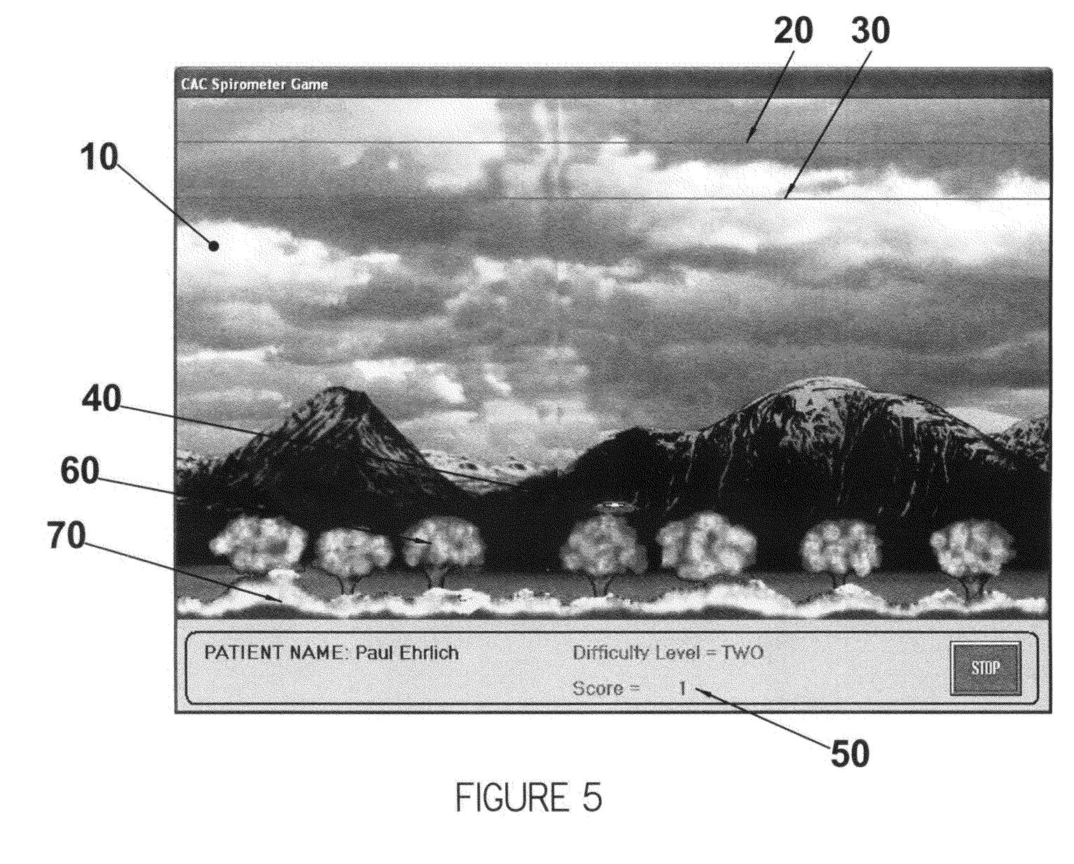

FIG. 5 shows a screenshot of a video monitor running the game-based Incentive Spirometer. The colorful background contains a mountainscape and clouds 10 that slowly move along the background of the scene. The rationale for having such natural looking scenery is so that the patient will feel more at ease when performing Incentive Spirometry therapy. If the patient does not utilize the therapy of utilizes it ineffectively, then it will do little good. When performing computer animation, it is common to have a multi-layered scene, where different parts of the scene move different rates, with the furthest scene—the mountainscape and clouds 10 moving at the slowest rate. In the tested version, the animated scene was composed of three individual scenes that all move at slightly different rates. The foremost scene 70 is showing scenery that signifies grass covered mounds. Since this part of the composite scenery is the foremost, it moves at the fastest rate. The next layer of scenery is the tree line 60, and this layer moves faster than the background but slower than the grass covered mounds 70. The effect of moving the background and foreground layers at different rates is to give the illusion of depth to the viewer. It is obvious to those skilled in the art that a moving background and an icon corresponding to the patient's inhalation or exhalation would be adequate to perform the same result, but having a multilayered foreground and background arrangement make the scene more pleasing to the eye. The little “space ship” 40 is an icon chosen to represent the patient's inhalation and exhalation. It does not have to be a space ship to work properly, as any object would work just as well. The space ship icon could easily have been a bird, an airplane, or a host of other objects. The object of the game-based Incentive Spirometer is to inhale at an appropriate rate for the typical three second period. When a patient inhales at the correct rate of approximately 1000 cubic centimeters per second (fro an adult), the little space ship 40 will rise up to the top of the screen. The goal is to hold the rate of inhalation of 1000 cubic centimeters per second, for a time period of at least three seconds. This will be indicated to the patient by having the space ship 40 rise upward between the two red lines. There is an upper red line 20 to signify the maximum rate of inhalation, and a lower red line 30 to signify the minimum rate of inhalation. When a patient successfully inhales at the proper rate, the space ship will rise to position itself between the upper 20 and lower 30 red lines. A scoring indication is displayed at the bottom of the screen 50. The value of the score 50 is not increasing because the patient failed to provide enough inhalation to cause the space ship to rise up between the upper red line 20 and the lower red line 30. The values of inhalation and exhalation rate, as well as the time intervals are automatically recorded onto the personal computers hard drive for later analysis by a care-giver or doctor. When the data is displayed in graphical form of rate vs. time, a graph can help a doctor or pulmonary specialist custom tailor a patient-specific therapy that will benefit the patient. Because the Incentive Spirometer is connected to a personal computer, a more adaptable therapy can be realized and varying degrees of difficulty can be implemented. With an Incentive Spirometer such as the type described in FIG. 1, there is no way to adjust the difficulty by making it more difficult. It can be made easier by holding the Incentive Spirometer at an angle, but it cannot be made more difficult. If a physically larger patient uses the same Incentive Spirometer as a very short adult, then each will not benefit from an effective Incentive Spirometry therapy. One former patient will find the effort exerted to be too little, while the latter patient will find the effort exerted to be too much.

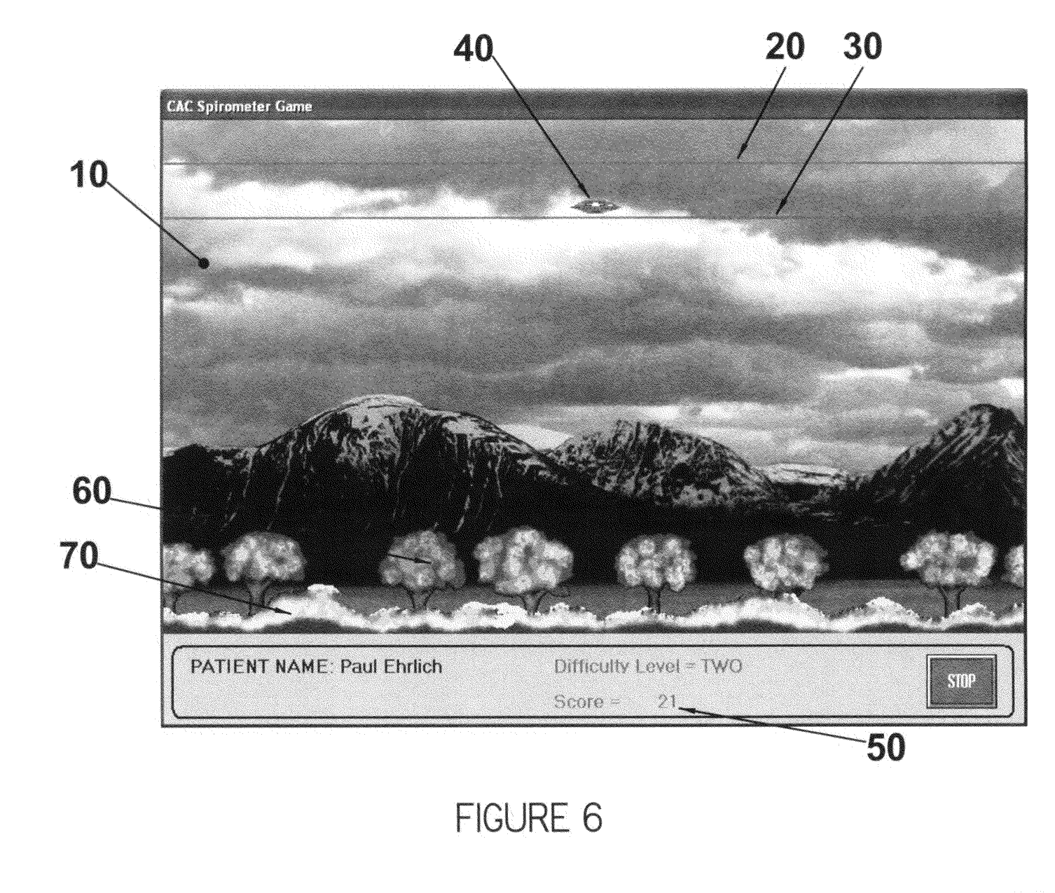

FIG. 6 shows a screenshot of a video monitor running the game-based Incentive Spirometer. The colorful background contains a mountainscape and clouds 10 that slowly move along the background of the scene. The rationale for having such natural looking scenery is so that the patient will feel more at ease when performing Incentive Spirometry therapy. If the patient does not utilize the therapy of utilizes it ineffectively, then it will do little good. When performing computer animation, it is common to have a multi-layered scene, where different parts of the scene move different rates, with the furthest scene—the mountainscape and clouds 10 moving at the slowest rate. In the tested version, the animated scene was composed of three individual scenes that all move at slightly different rates. The foremost scene 70 is showing scenery that signifies grass covered mounds. Since this part of the composite scenery is the foremost, it moves at the fastest rate. The next layer of scenery is the tree line 60, and this layer moves faster than the background but slower than the grass covered mounds 70. The little “space ship” 40 is an icon chosen to represent the patient's inhalation and exhalation. The object of the game-based Incentive Spirometer is to inhale at an appropriate rate for the typical three second period. When a patient inhales at the correct rate of approximately 1000 cubic centimeters per second (for an adult), the little space ship 40 will rise up to the top of the screen. The goal is to hold the rate of inhalation of 1000 cubic centimeters per second, for a time period of at least three seconds. This will be indicated to the patient by having the space ship 40 rise upward between the two red lines and be held there as long as they continue inhaling at the required rate. There is an upper red line 20 to signify the maximum rate of inhalation, and a lower red line 30 to signify the minimum rate of inhalation. When a patient successfully inhales at the proper rate, the space ship will rise to position itself between the upper 20 and lower 30 red lines. A scoring indication is displayed at the bottom of the screen 50. The value of the score 50 is increasing because the patient succeeded in providing enough inhalation to cause the space ship to rise up between the upper red line 20 and the lower red line 30. The values of inhalation and exhalation rate, as well as the time intervals are automatically recorded onto the personal computers hard drive for later analysis by a care-giver or doctor. When the data is displayed in graphical form of rate vs. time, a graph can help a doctor or pulmonary specialist custom tailor a patient-specific therapy that will benefit the patient. Because the Incentive Spirometer is connected to a personal computer, a more adaptable therapy can be realized and varying degrees of difficulty can be implemented. One method of setting the degree of difficulty is to vary the amount of time that a person has to inhale at a predetermined rate. It is much easier for a person to inhale or exhale at 1000 cubic centimeters per second for one second than it is for them to do so at five or six seconds. Having a time of five or six seconds would correspond to a higher difficulty than having a time of one second. As a patient enters the post operative period of their surgery, the degree of difficulty can be slowly increased as time passes. This would allow a patient to slowly increase the amount of effort exerted over time. Another means of changing difficulty is by allowing the maximum rate of inhalation to change from 1000 cubic centimeters per second to a higher value, such as 1100 or 1200 cubic centimeters per second. This would be what would be expected for a person of large physical stature, but above average for a person of smaller physical stature. By changing the rate displayed, which would correspond to the height of the small space ship 40 to reach a higher or lower position on the screen. The personal computer can scale the output of the airflow transducer to give a correspondingly higher or lower value of the raw value based upon software parameters. If the patient was a person of small physical stature, the difficulty setting could be configured to set the upper red line 20 lower on the computer screen so that the maximum rate of inhalation is lower than the average 1000 cubic centimeters per second. Likewise, the lower red line 30 that signifies the minimum rate of inhalation could be positioned lower on the computer screen. If both the upper 20 and lower 30 red lines are positioned at a height that is lower from the top of the computer screen, than the patient will be able to successfully position the small space ship 40 between the two lines, while producing an inhalation rate of that less than 1000 cubic centimeters per second. This would also be applied to a patient who is of a larger physical stature. The difficulty setting could be configured to set the upper red line 20 lower on the computer screen so that the maximum rate of inhalation is higher than the average 1000 cubic centimeters per second. Likewise, the lower red line 30 that signifies the minimum rate of inhalation could be positioned higher on the computer screen. If both the upper 20 and lower 30 red lines are positioned at a height that is closer to the top of the computer screen, than the patient must produce a higher rate of inhalation than the average 1000 cubic centimeters per second to be able to successfully position the small space ship 40 between the two lines. By utilizing the difficulty settings, a doctor of pulmonary specialist can compensate for a person of larger physical stature, as well as for a person of smaller physical stature. The result is that the patient (large or small) will effectively inflate and exhaust their lungs, regardless of the physical size of the lungs. If a typical commercially available Incentive Spirometer is utilized, the instrument would require a maximum of approximately 1000 cubic centimeters per second for a patient with a small set of lungs, as well as a patient with a much larger set of lungs. The described invention allows customization for a patient-specific therapy that would be much more effective than the traditional Incentive Spirometry therapy. A patient of larger physical stature would be able to produce an inhalation rate of over the typical 1000 cubic centimeters per second, while a patient of smaller physical stature, or elderly, may not be able to produce the typical rate of 1000 cubic centimeters per second. With the described invention, a personal computer would allow for a patient to accurately determine of their lungs are filling or exhausting completely. The baseline determination can be made by having the patient inhale and exhale over a period of approximately one minute while using the Incentive Spirometer that is connected to the personal computer. As the person completely exhales, the time and rate is logged onto the personal computers hard drive. Likewise, as the person inhales, the time and rate is logged onto the personal computers hard drive. By having a person completely exhale, then inhale several times, a baseline of what the person's maximum rate could be accurately determined. An effective Incentive Spirometry therapy is only effective if the patient is fully exercising their lungs. A patient of smaller physical stature may exert themselves too much while a patient of larger physical stature may under exert themselves. It should be obvious to those skilled in the art that a fully integrated Incentive Spirometry system could be built containing an LCD screen and a computer motherboard that would comprise a small, handheld unit that functions as an integrated Incentive Spirometer that would no longer require the connection to an external personal computer; however, the preferred embodiment of the described invention utilizes an external personal computer that is connected by means of a USB cable.

### REFERENCE NUMERALS

FIG. 1:

10 Main body of a commercially available Incentive Spirometer

20 Mouthpiece of the commercially available Incentive Spirometer

30 Flexible hose that connects the mouthpiece to the main body of the Incentive Spirometer

40 One of three individual plastic balls that are housed inside the main body of the Incentive Spirometer

FIG. 2:

10 Flexible hose that connects the mouthpiece to the main body of the Incentive Spirometer

20 Airflow transducer that converts flow rate into an analog signal

30 Connection that signifies the connection between the airflow transducer and the analog to digital converter

40 Analog to digital converter and microprocessor that converts the analog signal from the airflow transducer to a digital value

50 Connection that signifies the connection between the analog to digital converter and the interface circuitry

60 Interface circuitry that converts the digital values from the analog to digital converter to USB (Universal Serial Bus) protocol

70 Connection that signifies the connection between the interface circuitry and the personal computers USB port

80 USB port on the personal computer

90 Personal computer

100 Mouthpiece of the Incentive Spirometer that connects to the flexible tube

FIG. 3:

10 Patient that is utilizing the Incentive Spirometer

20 Flexible hose that connects the mouthpiece to the main body of the Incentive Spirometer

30 Integrated enclosure containing all the individual parts described in FIG. 2

40 USB Cable that connects the Incentive Spirometer to the personal computer (not shown)

50 Mouthpiece of the Incentive Spirometer that connects to the flexible tube

FIG. 4:

10 Personal computer

20 Table top

30 USB port on the personal computer

40 USB Cable that connects the Incentive Spirometer to the personal computer

50 Integrated enclosure containing all the individual parts described in FIG. 2

60 Flexible hose that connects the mouthpiece to the enclosure of the Incentive Spirometer

70 Mouthpiece of the Incentive Spirometer that connects to the flexible tube

FIG. 5:

10 Animation background displayed on a personal computer screen

20 Line that is displayed on the personal computer screen to indicate a maximum value of inhalation or exhalation rate

30 Line that is displayed on the personal computer screen to indicate a minimum value of inhalation or exhalation rate

40 Animated image of a small flying saucer space ship displayed on a personal computer screen

50 Graphical representation of the patients score displayed on a personal computer screen

60 Animated image of a tree line that is displayed in part of the foreground displayed on a personal computer screen

70 Animated image of grass covered mounds that are displayed in the foreground displayed on a personal computer screen

FIG. 6:

10 Animation background displayed on a personal computer screen

20 Line that is displayed on the personal computer screen to indicate a maximum value of inhalation or exhalation rate

30 Line that is displayed on the personal computer screen to indicate a minimum value of inhalation or exhalation rate

40 Animated image of a small flying saucer space ship displayed on a personal computer screen

50 Graphical representation of the patients score displayed on a personal computer screen

60 Animated image of a tree line that is displayed in part of the foreground displayed on a personal computer screen

70 Animated image of grass covered mounds that are displayed in the foreground displayed on a personal computer screen

What is claimed:

1. An incentive spirometry device comprising,
a removable flexible hollow tube with removable mouthpiece to allow for a person to exhale or inhale into an electronic sensor that is capable of measuring air flow rate and/or air volume;
an electronic sensor that is capable of accurately measuring air flow rate and/or air volume of inhaled or exhaled air by producing a corresponding electrical analog signal that relates directly to inhaled or exhaled air,
an electronic circuit that is capable of converting the analog signal produced from the electronic sensor into a digital signal,
an electronic processing circuit that contains a microprocessor that provides for external communication to a personal computer and is also capable of storing and processing the digitized electronic signal in such a way as to provide a means for real-time analysis of the rate of inhaled or exhaled air flow and comparing the real-time information to a stored set of parameters, and calculating whether the required inhaled or exhaled airflow is within designated parameters, below designated parameters, or above designated parameters, and also providing visual and/or audible feedback to the user, to indicate to the user that they are within, above, or below designated parameters,

2. An electronic processing circuit as in claim 1 where the electronic processing circuit transmits and receives electronic data to a personal computer comprising,
a wired connection that allows for communication to and from a personal computer via a USB (Universal Serial Bus), firewire, a serial COMM port utilizing EIA-232 protocol, a serial COMM port utilizing EIA-485 protocol, a parallel printer port, or Ethernet.
a wireless connection that allows for communication to and from a personal computer via a wireless interface utilizing either rf (radio frequency) or infrared signals.

3. A personal computer utilized in claim 2 where a proprietary program is run that produces a visual representation of a movable flying object, such as a spaceship, airplane, superhero, bird, balloon, or some other flying object, upon a moving background that is controlled by the inhaled or exhaled air flow of the user with the objective being to inhale or exhale at a great enough rate to hold the flying object within a designated minimum and maximum to allow for accelerated rehabilitation of a user. The personal computer will also store user data and allow for a doctor to analyze the information to determine if any progress is being made, and allow for an updated or modified program parameters to be sent to the personal computer via a direct download through a CD (compact disk), DVD (Digital Video Disk), USB Thumb drive, or by a wired or wireless connection through the Internet or World Wide Web (WWW).
